(19) United States
(12) Patent Application Publication
(10) Pub. No.: US 2026/0318197 A1
(43) Pub. Date: Oct. 15, 2026

(54) Elemental Rock Analysis For Offshore Drilling

(71) Applicant: Chevron U.S.A. Inc., San Ramon, CA (US)

(72) Inventor: Eric James Peavey, Magnolia, TX (US)

(21) Appl. No.: 19/463,401

(22) Filed: Jan. 29, 2026

Related U.S. Application Data
(60) Provisional application No. 63/752,617, filed on Jan. 31, 2025.

(51) Int. Cl. E21B 49/00; E21B 44/00; G01N 23/207; G01N 33/24

(52) U.S. Cl. CPC E21B 49/005; E21B 44/00; G01N 23/2076; G01N 33/24

(57) ABSTRACT

A computing system, method, and computer-readable medium includes a drill control system and an energy dispersive X-ray fluorescence analyzer onboard an offshore drilling rig. The energy dispersive X-ray fluorescence analyzer generates elemental data by analyzing rock cuttings from a wellbore in a subsea formation. The drill control system calculates mineralogy data and excess silica from the elemental data for the rock cuttings. The drill control system calculates an elastic modulus for the rock cuttings from the mineralogy data and the excess silica content. The drill control system adjusts at least one drilling parameter based upon one or more geomechanical properties, wherein the one or more geomechanical properties includes the elastic modulus of the rock cuttings.

### RELATED APPLICATIONS

The present application claims priority to U.S. Provisional Patent Application No. 63/752,617 filed Jan. 31, 2025, the entire content of which is incorporated herein by reference.

### TECHNICAL FIELD

Embodiments of the technology relate generally to determining properties of rock samples to improve subsea drilling techniques.

### BACKGROUND

Offshore production of hydrocarbons requires drilling and completing subsea hydrocarbon wells. Subsea hydrocarbon wells are sometimes located in challenging deepwater environments requiring substantial investments of time, planning, and resources to drill and complete the wells. As one example of a complex hydrocarbon well, subsea formations may require an unconventional well for producing oil or gas from the formation. Unconventional wells may involve high deviation (e.g., U-shaped) and/or horizontal drilling into rock formations with low permeability and porosity followed by hydraulic fracturing to produce oil or gas from the formation.

When drilling hydrocarbon wells (e.g., vertical, deviated, or horizontal), accurately estimating principal stresses, such as the minimum horizontal stress (Sₕₘᵢₙ), is important for maintaining wellbore stability and optimizing the drilling process. The minimum horizontal stress (Sₕₘᵢₙ) is equivalent to the closure pressure of fractures in the subsea formation. Factors affecting Sₕₘᵢₙ at a particular depth for a given wellbore path may include the depth of the wellbore (e.g., overburden or vertical stress), the rock lithology (e.g., elastic geomechanical properties), pore pressure within the formation, and magnitude and orientation of tectonic strains. In a subsea formation, the pore pressure and minimum horizontal stress can be particularly difficult to estimate accurately. Inaccurate estimations of pore pressure and Sₕₘᵢₙ can contribute to wellbore instability during drilling such as pressure kicks, substantial production of rock cavings (e.g., potentially indicating a wellbore undergoing collapse), and lost circulation events wherein drilling fluid leaks into the surrounding rock formation. Lost circulation events are a leading cause of disruption and non-productive downtime in the drilling process. Lost circulation events occurring during offshore drilling can result in substantial losses of resources and time.

It is widely known and accepted that X-Ray Fluorescence (XRF) analysis is sensitive to contaminants introduced into samples during sample collection and sample preparation processes. The accuracy of XRF analysis conducted on any material is also sensitive to matrix effects (that is, a homogenized matrix is preferable for a given prepped sample to improve reproducibility of results). Moreover, it is also understood that the signal recorded by an XRF scan of a material is related to the material density (e.g., the greater the material density, the more signal that should be recorded by a given XRF assay). It follows that every XRF measurement of a material contains density data inherent with its initial collection.

Accordingly, an improved understanding of the properties of the rock formation into which a hydrocarbon well is drilled can assist during drilling and can reduce the likelihood of occurrence of lost circulation events. As an example, U.S. Patent Application Publication Number 2023/0243803 describes techniques for determining properties of rock formations from the rock mineralogy. However, further improvements in determining the properties of subsea rock formations would be beneficial to offshore drilling.

### SUMMARY

The present disclosure is generally directed to controlling drilling parameters based upon geomechanical rock properties derived from analyzing rock cuttings. In one example, a method can comprise: a) generating elemental data by analyzing rock cuttings from a wellbore in a subsea formation, wherein the analysis is performed by an energy dispersive X-ray fluorescence analyzer onboard an offshore drilling rig; b) calculating mineralogy data from the elemental data, wherein the mineralogy data includes quartz content; c) calculating an excess silica content from the elemental data; d) calculating an elastic modulus for the rock cuttings from at least one of the excess silica content and the quartz content; and e) adjusting at least one drilling parameter based upon one or more geomechanical properties, wherein the one or more geomechanical properties includes the elastic modulus of the rock cuttings.

The foregoing embodiment can include one or more of the following additional features. The at least one drilling parameters can include one of a drill bit torque, a drill bit penetration rate, a drill bit azimuthal angle, a drill bit inclination angle, a drilling fluid density, and a surface back pressure. The excess silica can be determined from an amount of silica and an amount of Aluminum indicated by the elemental data. The foregoing method can further comprise: f) determining a principal stress from the elastic modulus for the rock cuttings; g) incorporating the principal stress into the one or more geomechanical properties; and h) adjusting the at least one drilling parameter based upon at least one of the elastic modulus and the principal stress. The principal stress can be a minimum horizontal stress for the subsea formation. The method can further comprise calibrating the principal stress using well data comprising at least one of shoe test data and lost circulation data.

The foregoing embodiments are non-limiting examples and other aspects and embodiments will be described herein. The foregoing summary is provided to introduce various concepts in a simplified form that are further described below in the detailed description. This summary is not intended to identify required or essential features of the claimed subject matter nor is the summary intended to limit the scope of the claimed subject matter.

### BRIEF DESCRIPTION OF THE DRAWINGS

The accompanying drawings illustrate only example embodiments of a method, system, and computer-readable media for adjusting one or more drilling parameters based upon geomechanical rock properties of a subsea formation. Therefore, the examples provided are not to be considered limiting of the scope of this disclosure. The principles illustrated in the example embodiments of the drawings can be applied to alternate methods and apparatus. Additionally, the elements and features shown in the drawings are not necessarily to scale, emphasis instead being placed upon clearly illustrating the principles of the example embodiments. In the drawings, the same reference numerals used in different embodiments designate like or corresponding, but not necessarily identical, elements.

FIG. 1 illustrates an overview of a method for adjusting one or more drilling parameters based upon geomechanical rock properties of a subsea formation in accordance with an example embodiment of the disclosure.

FIG. 2 and FIG. 3 illustrate details of a method for determining mineralogy data of subsea formation rock cuttings in accordance with an example embodiment of the disclosure.

FIG. 4 illustrates details of a method for determining elastic moduli of subsea formation rock cuttings for use in adjusting a drilling operation parameter in accordance with an example embodiment of the disclosure.

FIG. 5 and FIG. 6 illustrate details of a method for determining a principal stress for use in adjusting a drilling operation parameter in accordance with an example embodiment of the disclosure.

FIG. 7 illustrates operations of an optional method for applying a material density correction when applicable to the method of FIGS. 5 and 6.

FIGS. 8A, 8B, and 8C provide an example comparison of log-derived data and material density corrected data from XRF analysis of rock given example porosity and VSH assumptions, whereas FIG. 8D provides an example comparison of elastic moduli determined from log-derived and lab-calibrated data, with elastic moduli derived from XRF analysis of rock.

FIG. 9 illustrates example data comparing the determination of minimum horizontal stress from XRF analysis of rock and from traditional calculation methods.

FIG. 10 illustrates a scatterplot comparing minimum horizontal stress determined from XRF analysis of rock and from traditional calculation methods.

FIG. 11 illustrates a scatterplot comparing minimum horizontal stress determined from XRF analysis of rock and from traditional calculation methods.

FIG. 12 illustrates an example offshore drilling rig and drill control system in accordance with an example embodiment of the disclosure.

### DETAILED DESCRIPTION OF EXAMPLE EMBODIMENTS

The example embodiments discussed herein are directed to methods, systems, and computer-readable media for more accurately determining and/or providing an accurate, alternative determination of geomechanical properties of subsea rock formations for controlling drilling operations. As explained above, it can be challenging to accurately estimate the geomechanical properties of subsea rock formations and particularly the pore pressure and/or minimum horizontal stress (Sₕₘᵢₙ) of the formation. A more accurate understanding of the geomechanical properties of subsea rock formations allows for improved control of the drilling process and reduces the likelihood of occurrence of lost circulation events associated with wellbore stability problems. The techniques described herein for analyzing subsea rock cuttings in real-time on an offshore drilling rig allow for improvements to the drilling process. Such improvements are particularly beneficial in complex subsea drilling of both conventional and unconventional hydrocarbon wells.

Complex subsea drilling often is performed in shale rock. Shale rock typically comprises a combination of layered clay (i.e., silicate) mineral sheets held together by a distinct mineral matrix along with some percentage of mineral grains of various elemental composition and some percentage of diagenetic cements (e.g., comprised of one or more of calcite, dolomite, silica, or other mineral). Shale rock also includes organic material within its matrix and organic material and/or fluids (e.g., hydrocarbons and water) in its constituent pore networks. An improved understanding of the geomechanical properties of shale rock would be particularly beneficial to complex drilling in subsea rock formations.

Analyzing the subsea rock cuttings on the offshore drilling rig during drilling can provide valuable insights. In general, rock cuttings generated from the wellbore are produced by the coupled action of mechanical grinding of subsea formation by a drill bit and circulation of drilling fluid around the drill string and annulus of the pipe throughout a depth interval being actively drilled. The subsea rock formation cuttings produced from the wellbore at a particular depth take an amount of time to reach the surface that is dependent on factors like the drilling fluid density, drilling fluid circulation/pump rate (e.g., typically expressed in gallons per minute (gpm)), drilling fluid viscosity, and particle size of the cuttings. The lag time between rock cuttings being generated at depth and subsequently being produced by the wellbore at surface is often on the scale of hours. For example, given a penetration rate of the drill bit of 100 feet per hour, an initial depth of 20,000 ft, and a rock cuttings lag time of 5 hours from depth to surface, the formation cuttings from the depth 20,000 ft will reach the surface at a time when the drill bit penetrates 20,500 ft (thus a total of 500 ft of subsea formation are drilled in 5 hours). Thus, trends in the geomechanical rock properties determined from real-time elemental analysis of rock cuttings can be established and projected beyond the drill bit, particularly in cases where the rate of drill bit penetration is, for example, less than 100 ft/hr. For example, this condition (e.g., achieving a drilling rate less than 100 ft/hr in this case) may be satisfied while drilling geologic intervals that are known to produce wellbore stability problems within a particular field or basin, or while temporarily ceasing active drilling to circulate cuttings out of the wellbore (thus performing what is known as hole cleaning during the drilling process).

As described further below, the techniques provided herein include analyzing rock cuttings at the offshore drilling rig with a spectroscopic method known as energy dispersive X-ray fluorescence (ED-XRF) to identify the elements that make up the rock cuttings. The techniques provided herein also use improved methods for determining the amount of excess silica in the rock cuttings. These improvements provide a more accurate prediction of the geomechanical properties for the formation rock that is ahead of the drill bit. This more accurate prediction of the rock properties can be used to adjust a variety of drilling parameters to minimize the occurrence and severity of wellbore stability problems during the drilling process. As examples, drilling parameters that can be adjusted during the drilling process include the torque of the drill bit, the penetration rate of the drill bit, the drilling fluid density, the surface back pressure, and the azimuth and inclination angles of the drill bit.

While the example embodiments for controlling drilling based upon rock cuttings analysis are provided in the descriptions that follow, modifications to the embodiments described herein are within the scope of this disclosure. In the following paragraphs, particular embodiments will be described in further detail by way of example with reference to the drawings. In the description, well-known components, methods, and/or processing techniques are omitted or briefly described. Furthermore, reference to various feature(s) of the embodiments is not to suggest that all embodiments must include the referenced feature(s).

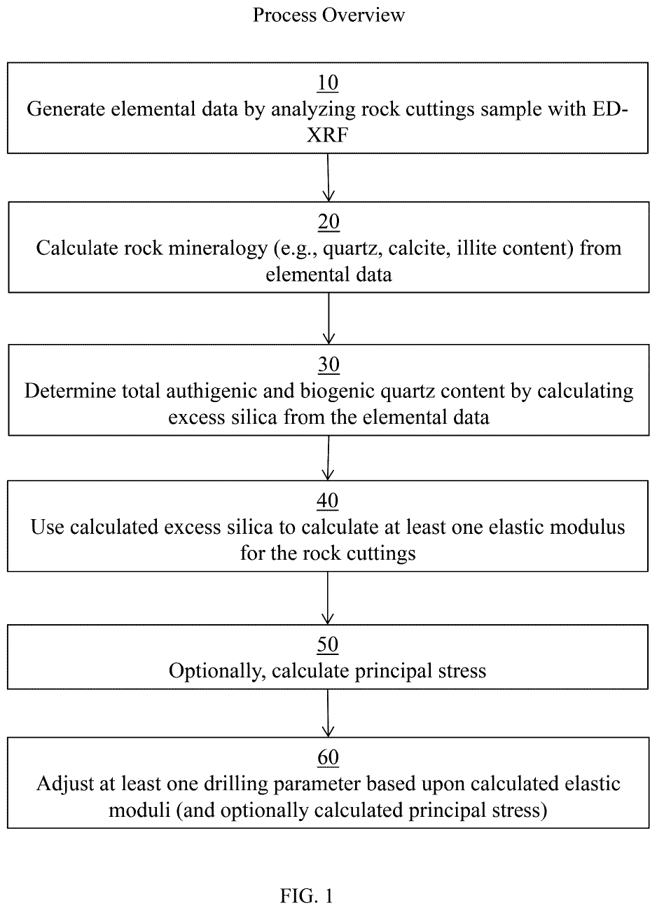

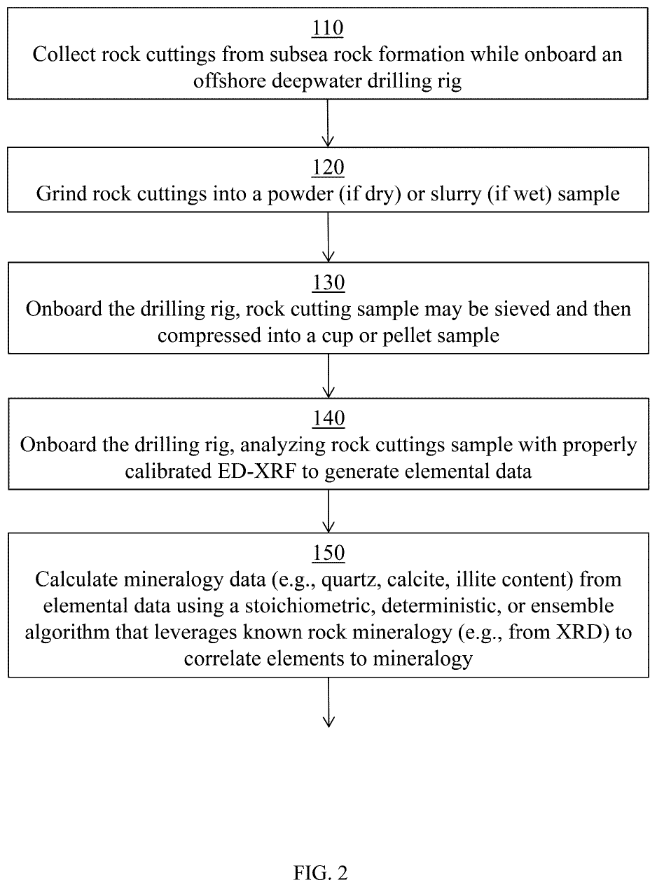

Referring now to FIG. 1, an overview of an example process is illustrated in accordance with the embodiments of the disclosure. Further details regarding the process of FIG. 1 will be described in connection with the more detailed processes illustrated in FIGS. 2-6 and described further below. Beginning with operation 10 of FIG. 1, an ED-XRF analyzer onboard the drilling rig scans rock cuttings that have been extracted from the subsea rock formation by the drilling rig. The ED-XRF analyzer generates elemental data regarding the elements in the rock cuttings. The elemental data can be used in real-time on the drilling rig to determine geomechanical properties of the subsea formation, which properties are used to inform the further drilling operations.

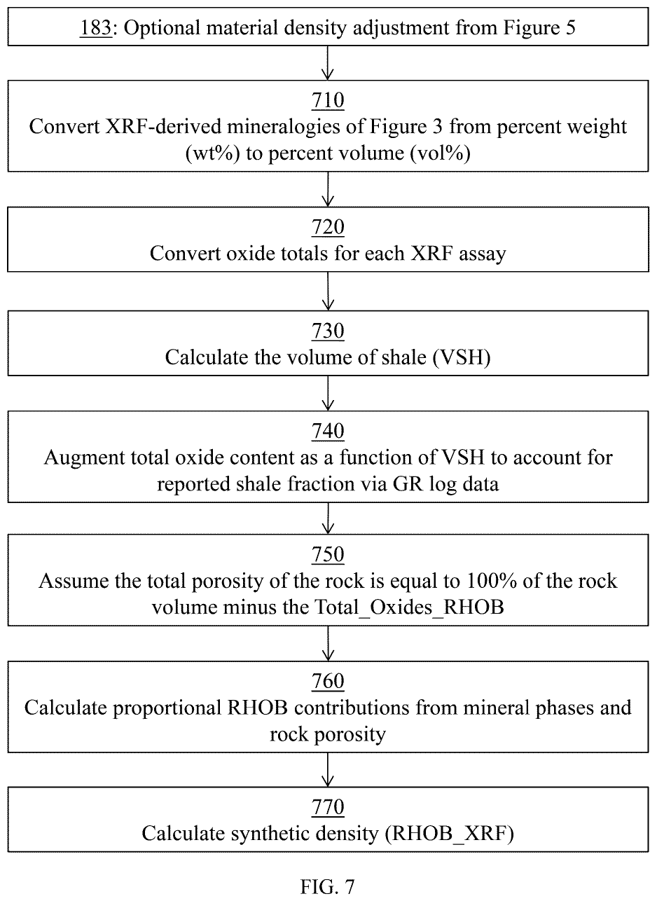

In operation 20 of FIG. 1, a geomechanical analysis service, such as that described further in association with FIG. 7 below, can calculate the mineralogy of the rock from the elemental data generated by the ED-XRF analyzer. As an example, the mineralogy can indicate the amount of quartz, calcite, and illite in the rock cuttings.

In operation 30 of FIG. 1, the geomechanical analysis service can calculate an amount of excess silica in the rock cuttings from the elemental data. The amount of excess silica is useful for determining the total amounts of authigenic quartz and biogenic quartz in the rock cuttings.

In operation 40 of FIG. 1, the calculated excess silica is used to calculate at least one elastic modulus for the rock cuttings. The elastic modulus can be Young's Modulus (horizontal and vertical) or Poisson's Ratio (horizontal and vertical).

Optionally, in operation 50 of FIG. 1, the geomechanical analysis service can calculate a principal stress for the subsea rock formation from the calculated elastic modulus. As one example of a principal stress, the minimum horizontal stress can be particularly useful for controlling drilling operations to avoid lost circulation events and potential wellbore instability.

In operation 60 of FIG. 1, a drill control system onboard the drilling rig can use the calculated elastic modulus, and optionally the calculated principal stress, to control and adjust drilling parameters of the drilling operation. Parameters that may be controlled or adjusted by the drill control system include drill bit torque, drill bit penetration rate, drill bit azimuthal angle, drill bit inclination angle, drilling fluid density, and surface back pressure (e.g., from an MPD system).

While the primary embodiments described herein are directed at analyzing subsea rock cuttings in real-time on the drilling rig as the cuttings reach the surface, the embodiments described herein also can be implemented in a laboratory setting prior to drilling. Specifically, geomechanical rock property trends for subsea rock formations can be determined prior to a well being drilled by performing the same rock cuttings analysis process presented herein in a laboratory. For the laboratory embodiment performed prior to drilling, rock cuttings samples are prepped in the laboratory in a similar manner as they would be at the offshore rig (e.g., using the methods described in FIG. 2). These rock cuttings would be sourced from a geologic offset well along local or regional pore pressure trends (e.g., field or basin scale) to an imminently active drilling well to determine elemental composition, mineralogy, and calculated geomechanical rock properties. The resulting calculated geomechanical rock property (e.g., elastic modulus) data can be quantitatively (e.g., graphically) compared with data collected from petrophysical tools, such as a sonic log, for a given depth range of analysis to validate the quality of the calculated geomechanical rock properties determined by this method.

FIGS. 2-6 provide a more detailed process for determining geomechanical properties of a subsea rock formation for use in controlling a drilling process. In alternate example embodiments, one or more of the operations illustrated in FIGS. 2-6 could be performed in parallel, performed in a different sequence, or eliminated. Furthermore, in alternate embodiments, other operations may be added to the method of FIGS. 2-6.

In operations 110-130 of FIG. 2, rock cuttings are prepared for analysis with an ED-XRF analyzer. Specifically, in operation 110 of FIG. 2, a drill bit of an offshore deepwater drilling rig generates rock cuttings from the subsea rock formation in which a well is being drilled. In operation 120, the rock cuttings are ground into a powder sample (if dry) or into a slurry sample (if wet). In operation 130, the rock cutting sample may be sieved and then compressed into a cup sample or pellet sample that is suitable for analysis.

In operation 140, an ED-XRF analyzer onboard the drilling rig can be used to scan the rock powder cup or pellet sample. Before analysis, the ED-XRF analyzer can be calibrated using elemental data generated from prior rock samples that have been verified via one or more independent laboratory analysis methods. The data generated by the ED-XRF may include values of a spectroscopic parameter, such as an emission or absorption signal, which can be mapped to amounts of particular elements present in the rock cuttings sample thereby providing the elemental data. The elemental data from the ED-XRF analysis is categorized into two families, namely, major elements (e.g., Al, Ca, Fe, K, Mg, Mn, Na, P, S, and Si) and trace elements lighter than, and including, Uranium. Unless otherwise noted or indicated by a particular context, major elements are those elements generally present in a sample with concentrations greater than 0.1% by weight, whereas trace elements are those elements present in a sample with concentrations less than 0.1% by weight.

As described further below in connection with FIG. 7, the elemental data from the ED-XRF can be stored in a database coupled to a drill control system on board the drilling rig. The drill control system can include computer-executable instructions, referred to as a geomechanical analysis service, that can perform calculations using the elemental data.

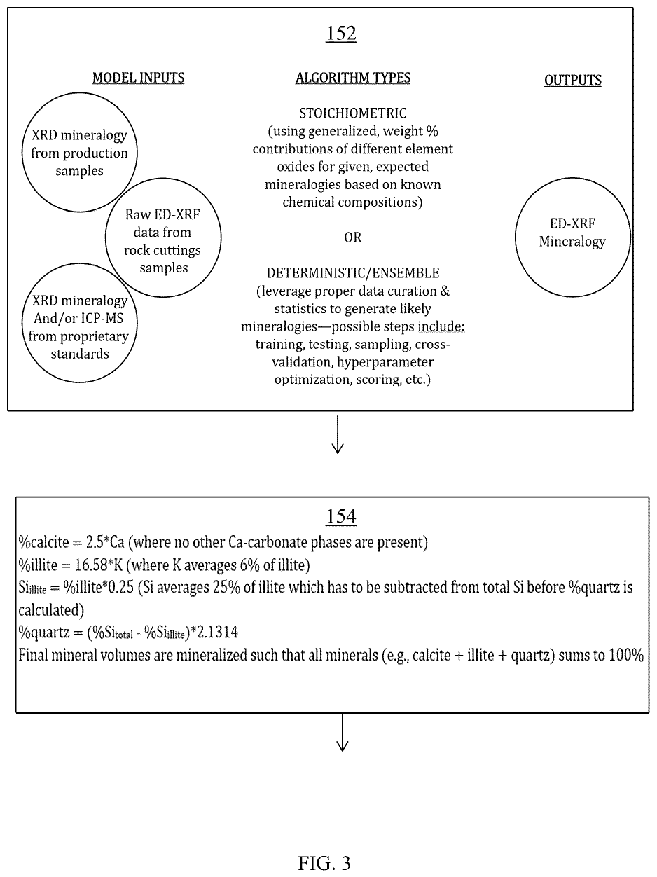

In operation 150 of FIG. 2, the geomechanical analysis service can calculate mineralogy data for the rock cuttings from the elemental data. The mineralogy data indicates the amount of quartz and other minerals, such as calcite and illite, present in the rock cuttings. The geomechanical analysis service utilizes pre-existing known data correlating elemental data and rock mineralogy to determine the mineralogy of the rock cuttings retrieved in the current drilling operation. The method for using known data to determine the mineralogy of rock cuttings can rely upon stoichiometric techniques, deterministic techniques, or an ensemble approach. The inputs and the available techniques for determining mineralogy of the rock cuttings are illustrated in greater detail in operations 152 and 154 of FIG. 3. As indicated in operation 152, in addition to the raw elemental data from the ED-XRF analysis of the rock cuttings samples, inputs can include X-ray diffraction (XRD) mineralogy data available from existing standards or from production samples, or Inductively Coupled Plasma Mass Spectrometry (ICP-MS) data available from existing standards.

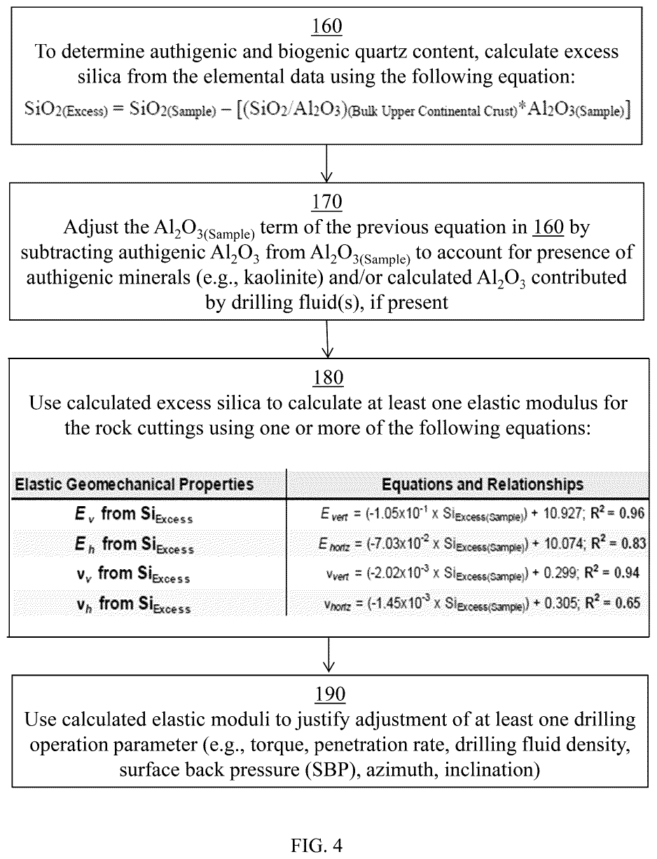

Referring now to FIG. 4, in operation 160, the geomechanical analysis service of the drill control system can determine an excess silica content in the rock cuttings sample. Prior approaches to determining an excess silica content from elemental data typically rely upon both major element and minor element amounts in the elemental data referenced above in connection with operation 140. For example, prior approaches would rely upon a ratio of a major element, such as Silicon, and a minor element, such as zirconium, or titanium, to determine the excess silica content in the rock cuttings sample. However, the prior approaches are susceptible to inaccuracies given the difficulties in accurately measuring trace elements and the non-uniform distribution of trace elements.

In contrast, the method of the present example calculates excess silica using only major elements detected in the elemental data. Using only the major elements in the elemental data provides for more accurate calculations. In the example of operation 160, the geomechanical analysis service calculates excess silica using the major elements Silicon and Aluminum and the equation provided in operation 160. Once the excess silica is calculated in operation 160, an adjustment is made in operation 170 to the equation from operation 160. Specifically, the excess silica calculated in operation 160 indicates the total amount of authigenic and biogenic quartz in the rock cuttings sample. Once the total amount of authigenic and biogenic quartz is determined, in operation 170, the Al₂O₃₍ₛₐₘₚₗₑ₎ term of the equation in 160 is adjusted by subtracting authigenic Al₂O₃ content to account for the presence of authigenic (e.g., diagenetic) mineral components of the rock cuttings composition. Additionally, the Al₂O₃₍ₛₐₘₚₗₑ₎ term can be adjusted to remove Al₂O₃ that is contributed by drilling fluids. With the adjustments described in operation 170, a more accurate calculation of the excess silica present in the rock cuttings is determined.

In operation 180, the geomechanical analysis service can calculate at least one elastic modulus for the rock cuttings using the excess silica content calculated in operations 160 and 170. The calculated elastic modulus can be Young's Modulus (vertical or horizontal) and/or Poisson's Ratio (vertical or horizontal), which can be initially calculated in accordance with the equations provided in operation 180. In the equations provided in operation 180, R² represents the coefficient of determination.

The one or more elastic moduli calculated in operation 180 are a geomechanical property providing insight into the subsea rock formation. In operation 190, the geomechanical analysis service can provide the one or more calculated elastic moduli to the drill control engine for use in controlling parameters of the drill bit. In certain embodiments, the geomechanical analysis service can use the one or more elastic moduli calculated in operation 180 in a machine learning model to predict the characteristics of the subsea rock formation ahead of the drill bit. In the example where a machine learning model is employed, the geomechanical analysis service can provide the predicted characteristics to the drill control engine. In operation 190, the drill control engine uses the elastic moduli and/or the predicted characteristics of the rock formation to adjust at least one drilling parameter of the drilling operation. Examples of the drilling parameters that can be adjusted include drill bit torque, drill bit penetration rate, drill bit azimuthal angle, drill bit inclination angle, drilling fluid density, and surface back pressure. As an example, if the calculated elastic moduli indicate the rock ahead of the drill bit is becoming relatively geomechanically compliant, the drill control engine can reduce the drilling fluid density and/or the surface back pressure accordingly to decrease the likelihood of inducing a lost circulation event.

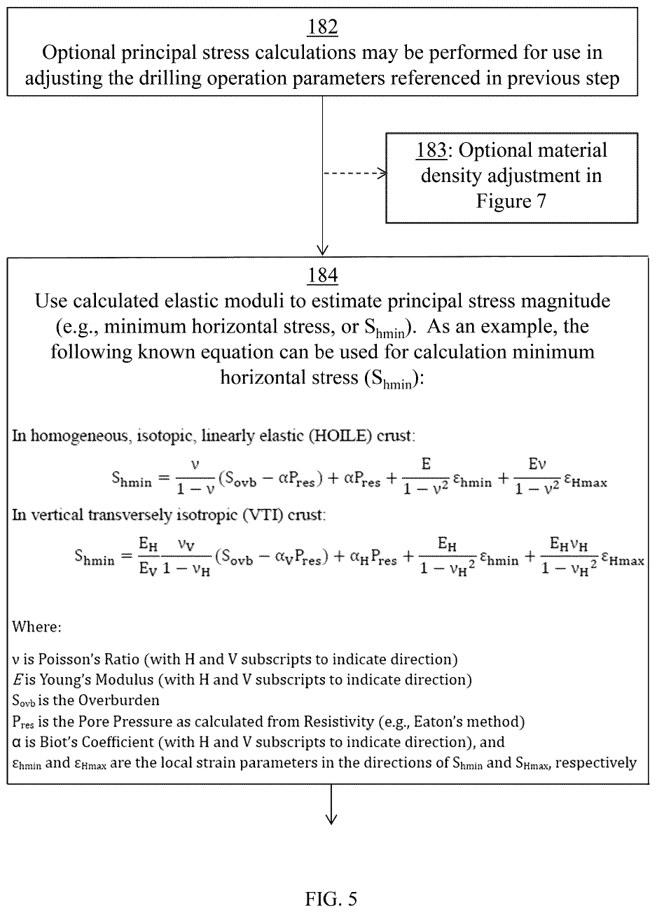

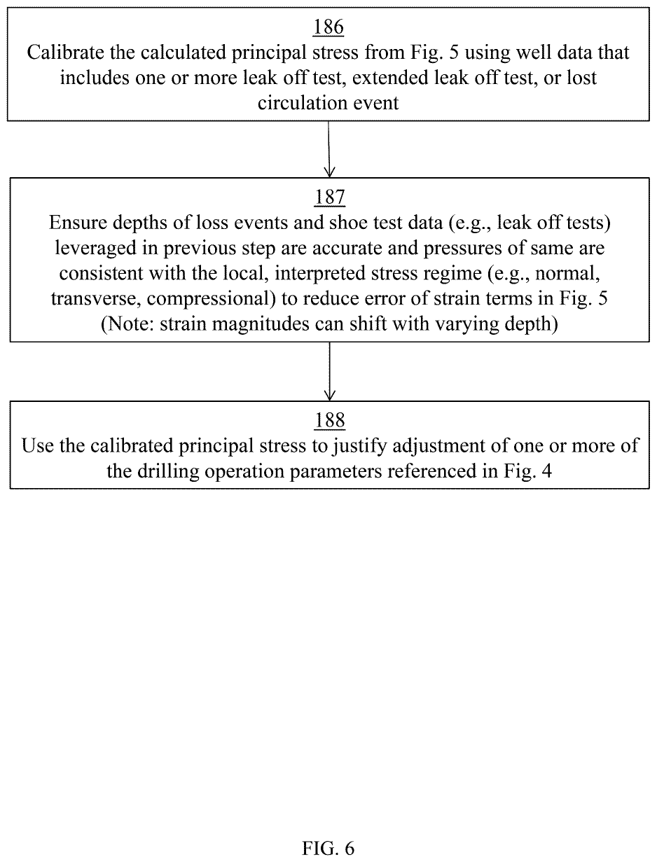

Referring now to FIGS. 5 and 6, additional optional calculations are described which can further enhance the control of the drilling operation. As indicated in operation 182, the geomechanical analysis service can calculate one or more principal stresses as additional geomechanical properties providing further insight into the characteristics of the subsea rock formation. Operation 183 provides an optional method for applying an adjustment to the calculations for varying densities of the basin in which the drilling is occurring. The optional method associated with operation 183 will be described in greater detail below in association with FIGS. 7-11.

Operation 184 provides an example whereby the geomechanical analysis service can calculate minimum horizontal stress (Sₕₘᵢₙ) for the subsea rock formation from the previously calculated elastic moduli. Sₕₘᵢₙ is equivalent to the closure pressure of fractures in the subsea formation and can provide an indication of pore pressure trend within the formation. Accordingly, Sₕₘᵢₙ is particularly useful in understanding the properties of the subsea rock formation for controlling drilling parameters. The process can return to operation 190 whereby the geomechanical analysis service provides the calculated Sₕₘᵢₙ to the drill control engine for use in adjusting one or more drilling parameters.

FIG. 6 provides additional operations that can be performed to achieve greater accuracy in calculating the geomechanical properties of the subsea rock formation. In operation 186, the geomechanical analysis service can calibrate the calculated principal stress from operation 184 using data associated with the well or appropriate offset wells. The data can include one or more of leak off test data, extended leak off test data, and lost circulation event data. In operation 187, the geomechanical analysis service can reduce error in the strain terms of the calculation in operation 184 by ensuring the loss events data and the shoe test data are accurate and consistent. At this point, at operation 188, the process can return to operation 190 whereby the geomechanical analysis service provides the calculated Sₕₘᵢₙ (as calibrated and corrected in operations 186 and 187) to the drill control engine for use in adjusting one or more drilling parameters.

Referring now to FIG. 7, the optional method previously referenced in operation 183 is described in greater detail. The additional operations of the method of FIG. 7 may be appropriate to make the elastic moduli equations of FIG. 4 more representative of a typical rock fabric for a given basin of interest. Depending on the basin within which offshore drilling is occurring, there may need to be modifications to the calculated elastic moduli to accurately estimate principal stress magnitude (e.g., minimum horizontal stress, or Sₕₘᵢₙ). The initial elastic moduli calculation equations presented in FIG. 4 assume the total volume of the rock is contributed solely by mineralogy (e.g., without pore networks or fluids within those pores). Thus, a material density correction may be applied to the initial mineralogies/total oxides calculated from a given rock cuttings sample for it to accurately reflect the true nature of the rock fabric, and thus accurately represent the various constituents contributing to the volume of the rock fabric (e.g., mineralogy, cements, pore networks, and pore fluids).

The operations of FIG. 7 may be performed to make the elastic moduli equations from FIG. 4 more representative of a typical rock fabric. In operation 710, the geomechanical analysis service can convert the existing XRF-derived mineralogies described above in FIG. 3 from precent weight (wt %) to percent volume (vol %) contributions to the rock fabric. The geomechanical analysis service can (i) calculate a mineral volume factor by dividing normalized weight percent mineral content by respective mineral density (in units of g/cc) and then (ii) calculate the total volume of mineral constituents. Next, the geomechanical analysis service can calculate intermediate volume/density factors by dividing each respective volume factor from (i) by the total mineral volume calculated in (ii).

In operation 720, the geomechanical analysis service can convert oxide totals derived in FIG. 3 above for each XRF assay. The geomechanical analysis service can sum the oxides reported by an ED-XRF scanner (e.g., reported major element oxides like SiO₂, Al₂O₃, TiO₂, Fe₂O₃ (and/or FeO), MnO₂, CaO, MgO, P₂O₅, K₂O, Na₂O, and SO₄). Note, SO₄ may be included or excluded from total oxide calculation, depending on the presence of BaSO₄ (barite) in mud (indicating degree of mud contamination needing correction) and presence of sulfide minerals (e.g., pyrite) in the rock fabric. The sum of oxides reported by an ED-XRF scanner should never equal 100% (e.g., 100.00) for any sample, except in the theoretical case of a sample comprised entirely of minerals and/or mineral cements that contains both zero porosity and zero organic matter. However, as explained in the Background section above, each sample analyzed by XRF contains density data inherent to the sample, which can be leveraged in cases where sample preparation/matrix homogenization is consistent for some number of samples used in a particular sequence of XRF analyses.

In operation 730, the geomechanical analysis service can calculate the volume of shale (“VSH”) for the rock samples using the appropriate equation from gamma ray data, acquired by either a total gamma ray calculated from XRF analysis using Uranium (U), Thorium (Th), and Potassium (K) elements, or using a spectral gamma ray collected by a downhole logging tool (e.g., near-bit gamma ray, LWD log, or wireline log). As an example, the Larionov equation curve 1 (index; linear relationship) can be used where:
i. VSH=I_{RA},
ii. I_{RA}=(GR_{log}−GR_{clean})/(GRₛₕₐₗₑ−GR_{clean}),
iii. GR=Gamma Ray,
iv. GR_{log}=GR value collected at a particular depth or point,
v. GR_{clean}=GR value collected at a depth assumed to be “clean sand”
vi. GRₛₕₐₗₑ=GR value collected at a depth assumed to be “shale”

Alternatively, VSH can be approximated using a strip log, built either by real-time lithology interpretations offered by mudloggers or by wellsite geologists during real time drilling operations. Although VSH calculated through this method would not be at as fine a resolution as that reported by log data, the VSH values calculated via strip log as a proportion of shale vs. total cuttings volume for a given depth remains comparable to that calculated from logs, and performs better in cases where shales are reported via cuttings to surface that are not as accurately captured by a log-derived VSH calculation alone.

As an additional alternative, VSH can be approximated by using both a combination of gamma ray data (either from LWD or total gamma ray, or both) and resistivity data (collected by resistivity tools often collecting data in tandem with gamma ray tools on a given borehole assembly), to further account for greater variety of lithologic variation observed within a given depth interval of interest.

As yet an additional alternative, VSH can be approximated by using a combination of gamma ray data (either from LWD or total gamma ray, or both), and volumetric mineralogy outputs of the mineralogic models from FIG. 2 and FIG. 3 converted to volumes determined in operations 710 and 720 from FIG. 7, adding mineralogic detail to the VSH calculation compared to traditional, log-based calculation methods.

In operation 740, the geomechanical analysis service can augment the total oxide content as a function of VSH to account for the reported shale fraction via GR log data provided by ED-XRF analysis or logging tools. The following equation provides the augmented total oxide content where RHOB=density:

Total_Oxides_RHOB=Total_Oxides+VSH*(100−Total_Oxides)

In operation 750, the geomechanical analysis service can determine the total porosity (Φ_{XRF}) of the rock sample as equal to 100% of the rock volume minus the Total_Oxides_RHOB determined in operation 740.

In operation 760, the geomechanical analysis service can calculate the proportional RHOB contributions from mineral phases and the rock porosity assuming the pore networks are filled with brine having a density of 1.03 g/cc.

Lastly, in operation 770, the geomechanical analysis service can calculate the synthetic density (RHOB_XRF) by summing the XRF-derived RHOB contributions to the rock fabric, including mineralogy, total porosity, and fluid density within that porosity (denoted in g) and apply a standard correction at the end of the equation of −0.10 to −0.20 g/cc to account for less Total Organic Carbon (TOC) content contained in those rock samples (this accounts for contributions of organic matter phases to overall rock volume for most rocks).

The following equations provide a summary of the calculations described in FIG. 7.
Start with modified equations for Deepwater assets:

Eᵥₑᵣₜ=0.25*((−1.05×10⁻¹*Si_{Excess(Sample)})+10.927)

E_{horiz}=0.25*((−7.03×10⁻²×Si_{Excess(Sample)})+10.074)

vᵥₑᵣₜ=(−2.02×10⁻³×Si_{Excess(Sample)})+0.349

v_{horiz}=(−1.45×10⁻³×Si_{Excess(Sample)})+0.355
Account for Material Density/Porosity

E_{vert[DW]}=Eᵥₑᵣₜ*((100−Φ_{XRF})/100)

E_{horiz[DW]}=E_{horiz}*((100−Φ_{XRF})/100)

v_{vert[DW]}=vᵥₑᵣₜ−0.5*(Φ_{XRF}/100)

v_{horiz[DW]}=v_{horiz}−0.5*(Φ_{XRF}/100)
Where:

Φ_{XRF}=100−XRF_{VSH_RHOB_FINAL}
And:

XRF_{VSH_RHOB_FINAL}=XRF_{TOTAL_OXIDES}+VSH*(100−XRF_{TOTAL_OXIDES})

The foregoing provides a correction that works well for drilling in the Gulf of Mexico. The foregoing provides a correction to the elastic moduli equations in FIG. 4 to better match lab data when making correlations of elastic moduli for rocks encountered in a particular basin of interest, whereby the total porosity calculated in operation 750 above is used to modify the calculations for the elastic moduli. For example, multiplying the Young's Modulus values by a factor of ¼ and adding 0.05 to the Poisson's Ratios equations helps to calibrate the equations to help them work effectively in basins not targeting unconventional reservoirs.

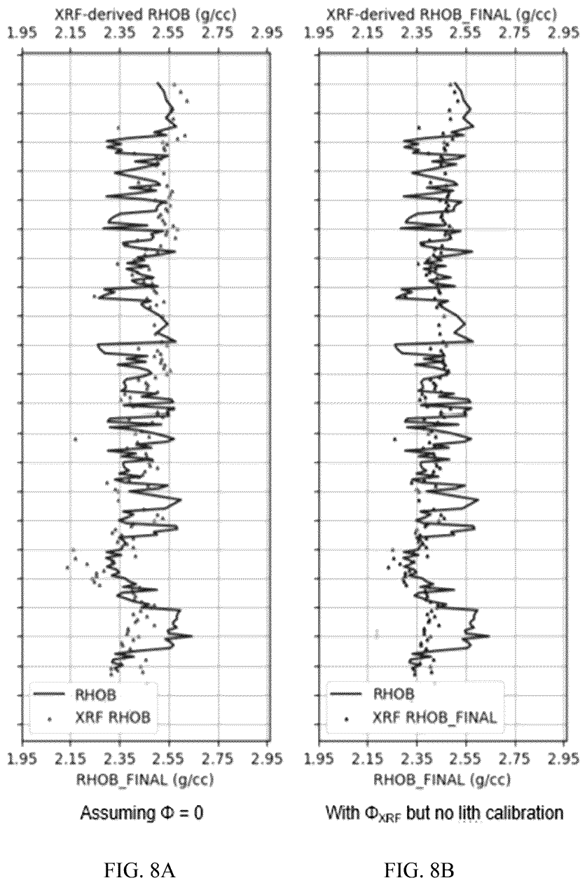

FIGS. 8A, 8B, and 8C, provide an example of a comparison of XRF-generated density data vs. log data given different rock property assumptions, and FIG. 8D provides an example of a comparison of at least one elastic moduli vs. log-derived and lab-calibrated data. Specifically, FIGS. 8A, 8B, and 8C provide log comparisons showing the difference between a density (RHOB) log and XRF-generated density data, with assumptions including, porosity is zero, Φ_{XRF} is non-zero but XRF data are not lith-calibrated, Φ_{XRF} is non-zero and the XRF data are calibrated to volume of shale. In FIG. 8D, PR_CALIB is a Poisson's Ratio calibrated to lab data and offset well logs for a particular stratigraphic interval of interest, and XRF PR is XRF-derived Poisson's Ratio. The XRF-derived Poisson's Ratio is determined from the V_{vert[DW]}equation provided above in association with FIG. 7.

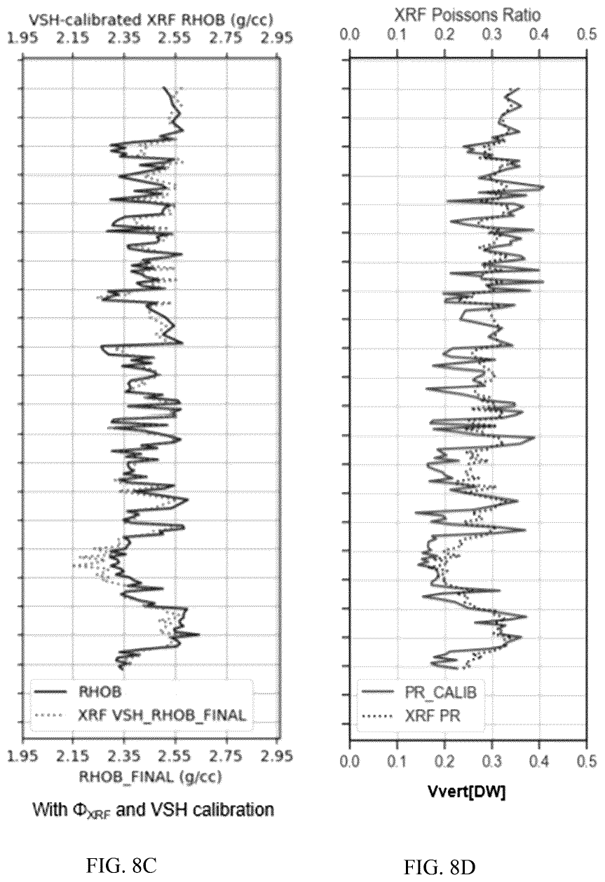

FIG. 9 provides visual comparisons of XRF-derived Sₕₘᵢₙ determined from rock cuttings (RT PLC_CALIB; calibrated to shale volume or VSH; dashed curve) compared with alternative Sₕₘᵢₙ calculations using lithology interpreted from wellsite geologists for shale volume (RT PLC—WSG; dotted curve) and lithology interpreted from mudloggers from a vendor to understand shale volume (RT PLC; dashed-dotted curve), with all curves compared against the pre-drill Sₕₘᵢₙ calculation (solid curve). The solid Sₕₘᵢₙ curve represents the “answer” determined by existing workflows in calculating Sₕₘᵢₙ from log data and calibrated to strain data provided by shoe tests, dynamic FITs, and/or lost circulation events encountered while drilling. Differences in Sₕₘᵢₙ calculations may be observed for more homogenous lithologies and/or for depth intervals of interest where increased compaction or effective stress is observed over time which may represent a “final state” of a rock in a depletion scenario.

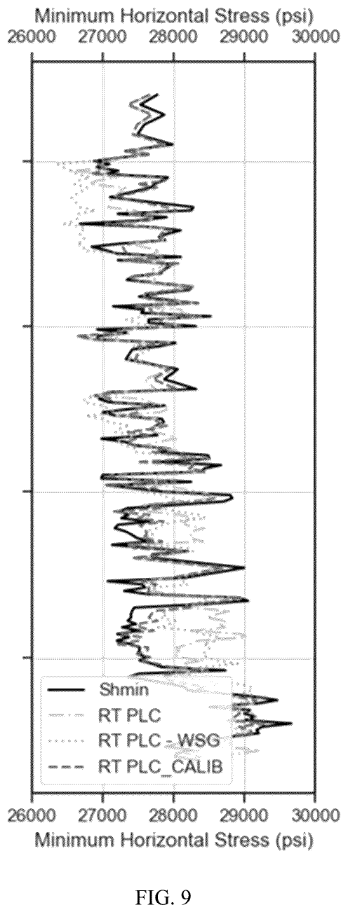

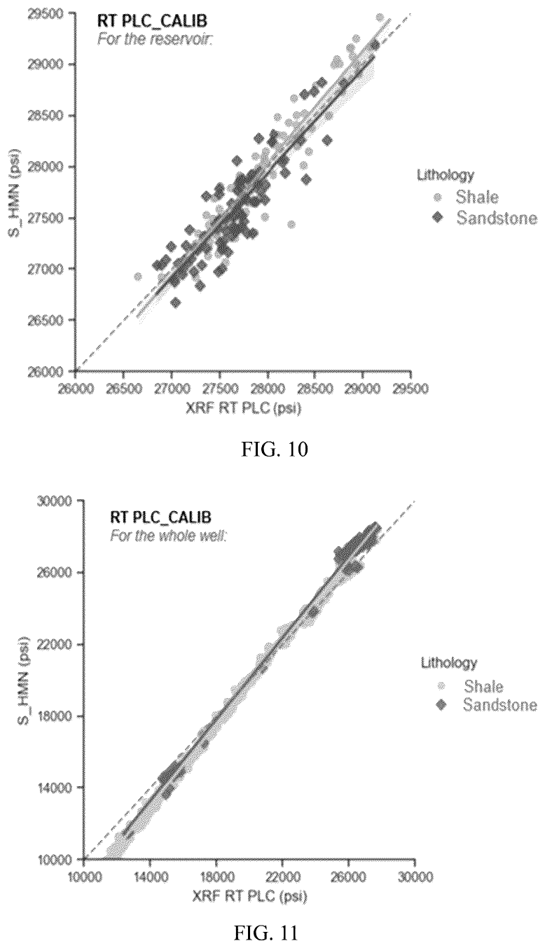

The scatterplots in FIGS. 10 and 11 visualize the data from the solid curve (Sₕₘᵢₙ) vs. the data from the RT PLC_CALIB, or XRF-derived Sₕₘᵢₙ, curve demonstrating good agreement between traditional methods for calculating Sₕₘᵢₙ and using XRF analysis of rock cuttings from a well in real time to accomplish a similar result. Note the slope of the data across an entire well doesn't quite land on the 1:1 line—this is because calibration points for stress state were applied only to the center depth of the curve (closest to the dashed 1:1 line) at a depth different than the result when the Sₕₘᵢₙ is calibrated to a particular depth interval. The result is an Sₕₘᵢₙ interpretation that must have calibrated strain parameters at different depth intervals, informed by offset LOTs, XLOTs, DFITs, lost circulation events, and real time results of the given well having its rock cuttings analyzed by XRF during drilling operations.

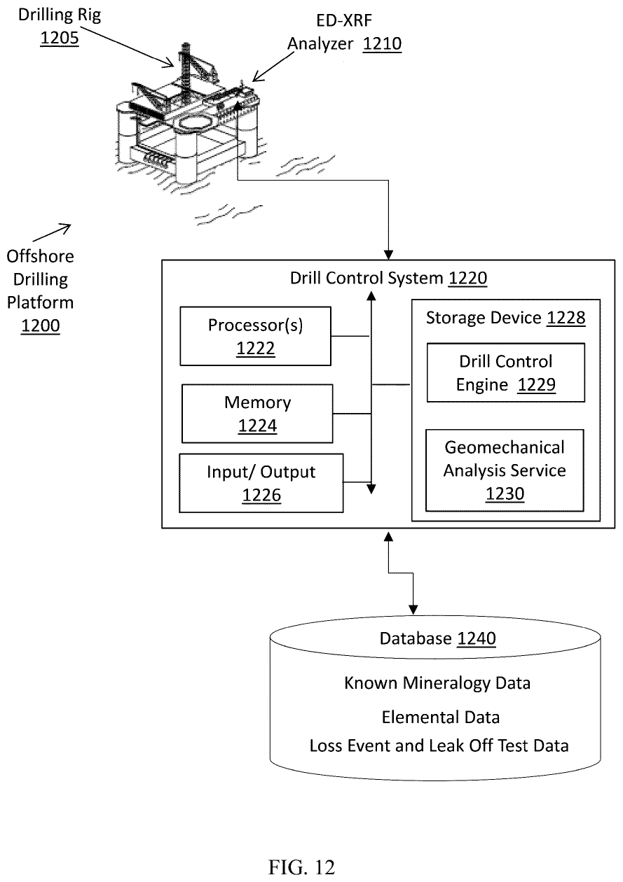

Referring now to FIG. 12, an example offshore drilling rig 1205 at the surface of a body of water is illustrated. The offshore drilling rig 1205 can include equipment such as one or more cranes, a rack of pipe segments, and a derrick that supports the drill string extending below the rig and into the subsea rock formation. At the top surface of the rig 1205, an ED-XRF analyzer 1210 is used to analyze the rock cuttings retrieved from the subsea rock formation. A drill control system 1220 receives from the ED-XRF analyzer elemental data describing the elements that make up the rock cuttings.

As is commonly known for control systems, the drill control system 1220 includes one or more processors 1222, memory 1224, and input/output interfaces 1226. The storage device 1228 can be an integrated component of the drill control system 1220, as illustrated in FIG. 12, or it can be external to the drill control system. In addition to an operating system, the storage device 1228 includes computer-executable instructions for retrieving data from a database 1240, performing calculations, and generating commands and recommendations for the operation of the drilling rig's drill bit. The computer-executable instructions can include a drill control engine 1229 which generates commands for operating the drilling rig's drill bit, including commands associated with drilling parameters. The computer-executable instructions also can include a geomechanical analysis service 1230 that uses data from the database 740 to calculate geomechanical properties associated with the subsea rock formation into which the well is being drilled.

The database 1240 stores the elemental data received from the ED-XRF analyzer 710. In addition, the database 1240 can store known mineralogy data that is used to calculate the mineralogy of the subsea rock formation from the elemental data for the rock cuttings retrieved by the drilling rig 1205. The database 1240 also can store data associated with the drilling operation, such as loss event data and leak off test data, which can be used in calibrating principal stress calculations.

The computing components illustrated in FIG. 12 are merely illustrative examples and in alternate embodiments certain of the computing components can be combined, simplified, or distributed in a different manner. For example, while the geomechanical analysis service 1230 is illustrated in FIG. 12 as one or more software modules stored in storage device 1228 of the drill control system 1220, the geomechanical analysis service 1230 also can be implemented as a service available on remote computing devices, such as the cloud, which service can be accessed to analyze data associated with the subsea rock formation and provide recommendations for managing the drilling operation. Additionally, while the geomechanical analysis service 1230 is described herein as an integrated software service, in other embodiments the service can be distributed across multiple services or computing systems. Furthermore, in certain embodiments the geomechanical analysis service 1230 can include a machine learning model that is trained using historical rock data and well data to predict rock mineralogy or geomechanical properties of the subsea rock formation that is currently being drilled.

### General Information Regarding Computing Systems

As described in connection with FIGS. 1-12, some or all of the processing operations described in connection with the foregoing systems and methods can be performed by computing systems such as a personal computer, a desktop computer, a computer server, or cloud computing systems. As explained previously, certain operations of the foregoing methods can be performed by a combination of computing systems.

The computing systems used in the foregoing embodiments can include typical components such as one or more processors, memories, input/output devices, and storage devices. The components of the computing systems can be interconnected, for example, by a system bus or by communication links. The components of the previously described computing systems are not exhaustive.

The one or more processors can be one or more hardware processors and can execute computer-readable instructions, such as instructions stored in a memory. The processor can be an integrated circuit, a central processing unit, a multi-core processing chip, an SoC, a multi-chip module including multiple multi-core processing chips, or other hardware processor in one or more example embodiments. The hardware processor is known by other names, including but not limited to a computer processor, a microprocessor, and a multi-core processor.

The memory can store information including computer-readable instructions and data. The memory can be cache memory, a main memory, and/or any other suitable type of memory. The memory is a non-transitory computer-readable medium. In some cases, the memory can be a volatile memory device, while in other cases the memory can be a non-volatile memory device.

The storage device can be a non-transitory computer-readable medium that provides large capacity storage for a computing system. The storage device can be a disk drive, a flash drive, a solid state drive, or some other type of storage device. In some cases, the storage device can be a database that is remote from the computing system. The storage device can store operating system data, file data, database data, algorithms, and software modules, as examples.

### Assumptions and Definitions

For any figure shown and described herein, one or more of the components may be omitted, added, repeated, and/or substituted. Accordingly, embodiments shown in a particular figure should not be considered limited to the specific arrangements of components shown in such figure. Further, if a component of a figure is described but not expressly shown or labeled in that figure, the label used for a corresponding component in another figure can be inferred to that component. Conversely, if a component in a figure is labeled but not described the description for such component can be substantially the same as the description for the corresponding component in another figure.

With respect to the example methods described herein, in alternate embodiments, certain steps of the methods may be performed in a different order, may be performed in parallel, or may be omitted. Moreover, in alternate embodiments additional steps may be added to the example methods described herein. Accordingly, the example methods provided herein should be viewed as illustrative and not limiting of the disclosure.

Terms such as “first” and “second” are used merely to distinguish one element (or state of an element) from another. Such terms are not meant to denote a preference and are not meant to limit the embodiments described herein. In the example embodiments described herein, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

The terms “a,” “an,” and “the” are intended to include plural alternatives, e.g., at least one. The terms “including”, “with”, and “having”, as used herein, are defined as comprising (i.e., open language), unless specified otherwise.

Values, ranges, or features may be expressed herein as “about”, from “about” one particular value, and/or to “about” another particular value. When such values, or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In another aspect, use of the term “about” means ±20% of the stated value, ±15% of the stated value, ±10% of the stated value, ±5% of the stated value, ±3% of the stated value, or ±1% of the stated value.

Although embodiments described herein are made with reference to example embodiments, it should be appreciated by those skilled in the art that various modifications are well within the scope of this disclosure. Those skilled in the art will appreciate that the example embodiments described herein are not limited to any specifically discussed application and that the embodiments described herein are illustrative and not restrictive. From the description of the example embodiments, equivalents of the elements shown therein will suggest themselves to those skilled in the art, and ways of constructing other embodiments using the present disclosure will suggest themselves to practitioners of the art. Therefore, the scope of the example embodiments is not limited herein.

What is claimed is:

1. A method comprising:
generating elemental data by analyzing rock cuttings from a wellbore in a subsea formation, wherein the analysis is performed by an energy dispersive X-ray fluorescence analyzer onboard an offshore drilling rig;
calculating mineralogy data from the elemental data, wherein the mineralogy data includes a quartz content;
calculating an excess silica content from the elemental data;
calculating an elastic modulus for the rock cuttings from at least one of the excess silica content and the quartz content; and
adjusting at least one drilling parameter based upon one or more geomechanical properties, wherein the one or more geomechanical properties includes the elastic modulus of the rock cuttings.

2. The method of claim 1, wherein the at least one drilling parameter is one of a drill bit torque, a drill bit penetration rate, a drill bit azimuthal angle, a drill bit inclination angle, a drilling fluid density, and a surface back pressure.

3. The method of claim 1, wherein the mineralogy data is calculated using a stoichiometric algorithm, a deterministic algorithm, or an ensemble algorithm.

4. The method of claim 1, wherein the excess silica is determined from an amount of silica and an amount of Aluminum indicated by the elemental data.

5. The method of claim 1, further comprising:
determining a principal stress from the elastic modulus for the rock cuttings;
incorporating the principal stress into the one or more geomechanical properties; and
adjusting the at least one drilling parameter based upon at least one of the elastic modulus and the principal stress.

6. The method of claim 5, further comprising applying a correction to the elastic modulus for the rock cuttings by determining a material density correction for the mineralogy data.

7. The method of claim 5, further comprising calibrating the principal stress using well data comprising at least one of shoe test data and lost circulation data.

8. A system onboard an offshore drilling rig, the system comprising:
an energy dispersive X-ray fluorescence analyzer; and
a drill control system, the drill control system comprising a processor, a memory, and a storage device, wherein the storage device comprises computer-executable instructions that when executed by the processor perform a method comprising:
receiving elemental data by analyzing rock cuttings from a wellbore in a subsea formation, wherein the analysis is performed by the energy dispersive X-ray fluorescence analyzer;
calculating mineralogy data from the elemental data, wherein the mineralogy data includes a quartz content;
calculating an excess silica content from the elemental data;
calculating an elastic modulus for the rock cuttings from at least one of the excess silica content and the quartz content; and
adjusting, by the drill control system, at least one drilling parameter based upon one or more geomechanical properties, wherein the one or more geomechanical properties includes the elastic modulus of the rock cuttings.

9. The system of claim 8, wherein the at least one drilling parameter is one of a drill bit torque, a drill bit penetration rate, a drill bit azimuthal angle, a drill bit inclination angle, a drilling fluid density, and a surface back pressure.

10. The system of claim 8, wherein the mineralogy data is calculated using a stoichiometric algorithm, a deterministic algorithm, or an ensemble algorithm.

11. The system of claim 8, wherein the excess silica is determined from an amount of silica and an amount of Aluminum indicated by the elemental data.

12. The system of claim 8, wherein the method further comprises:
determining a principal stress from the elastic modulus for the rock cuttings;
incorporating the principal stress into the one or more geomechanical properties; and
adjusting, by the drill control system, the at least one drilling parameter based upon at least one of the elastic modulus and the principal stress.

13. The system of claim 12, further comprising applying a correction to the elastic modulus for the rock cuttings by determining a material density correction for the mineralogy data.

14. The system of claim 12, wherein the method further comprises calibrating the principal stress using well data comprising at least one of shoe test data and lost circulation data.

15. A non-transitory computer-readable medium of a drill control system onboard an offshore drilling rig, the non-transitory computer-readable medium comprising computer-executable instructions that when executed by a hardware processor perform a method comprising:
receiving elemental data generated by analyzing rock cuttings from a wellbore in a subsea formation, wherein the analysis is performed by an energy dispersive X-ray fluorescence analyzer;
calculating mineralogy data from the elemental data, wherein the mineralogy data includes a quartz content;
calculating an excess silica content from the elemental data;
calculating an elastic modulus for the rock cuttings from at least one of the excess silica content and the quartz content; and
adjusting, by the drill control system, at least one drilling parameter based upon one or more geomechanical properties, wherein the one or more geomechanical properties includes the elastic modulus of the rock cuttings.

16. The non-transitory computer-readable medium of claim 15, wherein the at least one drilling parameter is one of a drill bit torque, a drill bit penetration rate, a drill bit azimuthal angle, a drill bit inclination angle, a drilling fluid density, and a surface back pressure.

17. The non-transitory computer-readable medium of claim 15, wherein the excess silica is determined from an amount of silica and an amount of Aluminum indicated by the elemental data.

18. The non-transitory computer-readable medium of claim 15, wherein the method further comprises:
determining a principal stress from the elastic modulus for the rock cuttings;
incorporating the principal stress into the one or more geomechanical properties; and
adjusting, by the drill control system, the at least one drilling parameter based upon at least one of the elastic modulus and the principal stress.

19. The non-transitory computer-readable medium of claim 18, further comprising applying a correction to the elastic modulus for the rock cuttings by determining a material density correction for the mineralogy data.

20. The non-transitory computer-readable medium of claim 18, wherein the method further comprises calibrating the principal stress using well data comprising at least one of shoe test data and lost circulation data.
